(19) United States
(12) Patent Application Publication
(10) Pub. No.: US 2005/0220712 A1
(43) Pub. Date: Oct. 6, 2005

(54) Method for determining mucosal neutrophil counts in neutropenia patents

(76) Inventors: Daniel G. Wright, Portland, ME (US); William J. Palin, Portland, ME (US); Turner Nathan, Portland, ME (US)

Correspondence Address: MARY HELEN SEARS;THE M.H. SEARS LAW FIRM, CHTD., Suite 800, 910 Seventeenth Street, N.W., Washington, DC, 20006, US

(21) Appl. No.: 10/814,646

(22) Filed: Apr. 1, 2004

(51) Int. Cl. A61K 49/00; C12Q 1/00

(52) U.S. Cl. 424/9.6; 435/4

(57) ABSTRACT

Based on recent investigations showing iatrogenic, profound neutropenia (and the fever spikes that often accompany it, which must be medicated immediately to avoid the risk of life-threatening infection) can most accurately be monitored by obtaining daily mucosal neutrophil counts from the patient's oral mucosa rather than obtaining daily counts of the patient's blood neutrophils as in the past, a mouth wash method has been developed for collecting muscosal neutrophils. The mouth wash samples so collected are delivered directly, or in aqueous dilution to a sample pad supported on a strip which sample pad has deposited thereon reagents enabling a colorimetric, fluorescent or chemiluminescent assay of the quantity of an enzyme characteristic of human neutrophils that is present in the sample. This measured quantity can be correlated to mucosal neutrophil count. The method shows outstanding sensitivity, precision and accuracy relative to microscopic methods of counting mucosal neutrophils.

### INTRODUCTION

The present invention relates to a quick, easy-to-use diagnostic test for monitoring the severity of neutropenia conditions in human patients.

### BACKGROUND OF THE INVENTION

Neutropenia is a serious deficiency in humans of infection-fighting white blood cells. Its severity has heretofore been tracked by periodic (usually daily) determinations of the individual blood neutrophil count of each patient who is in danger of developing the so-called “profound” or iatrogenic form of this deficiency, which exposes the patient to the danger of developing acute, often life-threatening, bacterial or fungal infection.

Profound or iatrogenic neutropenia, in which the patient is so exposed, most often occurs in two groups of patients, namely (a) individuals who have been subjected to high dose, cytotoxic, anticancer chemotherapy and (b) HIV patients who have either natural or acquired resistance to the protease inhibitors widely used in antiretroviral therapy.

Both groups of patients were normally maintained in the hospital environment when aggressive chemotherapy and antiretroviral therapy were each relatively new. In the case of high dose chemotherapy, it is currently very widely used in the treatment of many cancers, including carcinomas of various organs, such as breast lung and intestinal carcinomas, and in blood malignancies such as leukemias, lymphomas and myelomas. Three decades of experience with aggressive, high dose anticancer therapy has led to adjunct improvements in anti-emetic use, antibiotic prophylaxis, availability of myelopoietic (including hematopoietic) growth factors and use of autologous blood stem cell transplantation. In many cases, these adjunct improvements have enabled the administration of high dose anticancer chemotherapy to be moved from the hospital to outpatient settings, where daily blood count monitoring is not easily accomplished. Nevertheless, it remains critical to success of the therapy and the general health of the patient that, since chemotherapy inevitably arrests normal blood cell production and necessarily causes some degree of neutropenia in every patient, there be a reliable system in place for detecting the onset of the profound (or iatrogenic) stage of neutropenia and immediately medicating the patient with intravenous broad spectrum antibiotics to forestall aggravated bacterial or fungal infection.

Studies of neutrophil kinetics showed, as early as the 1980's, that neutrophil blood counts do not uniformly and consistently reflect the neutrophil population on extravascular surfaces where the principal work of repelling infectious bacteria and fungi is effected by neutrophils.

One of the co-inventors of this application, Daniel G. Wright, M.D. participated with A. I. Meierovics and J. M Foxley in a study, the results of which they published in “Assessing the Delivery of Neutrophils to Tissues in Neutropenia”, Blood 67, pp. 1023-1030 (1986), wherein they developed a reproducible method of quantifying oral mucosal neutrophils which they collected with an oral saline rinse, stained with acridine orange and counted using fluorescence microscopy. Their tests encompassed a group of healthy patients, a group of chronically neutropenic individuals and another group with acute neutropenia following anticancer chemotherapy. In each group, mucosal neutrophil count was compared with blood neutrophil count. The authors concluded that mucosal neutrophil counts in profoundly neutropenic individuals and their differences from blood neutrophil counts observed in the same individuals in samples taken at the same time, deserved further study.

Coinventor Wright, with G. Akpek and R. D. Knight, has now conducted further studies reported in “Use of Oral Mucosal Neutrophil Counts to Detect the Onset and Resolution of Profound Neutopenia Following High-Dose Myelosuppressive Chemotheraphy”, Am. J. Hematol 72, 13-19 (2003) as to which these authors concluded, inter alia, that oral mucosal neutrophil counts define nadirs of neutropenia more accurately than blood neutrophil counts do—and that the onset and resolution of neutropenic fever (defined as a body temperature above 101° F.) coincideds more precisely with mucosal neutrophil counts than with blood neutrophil counts. In the study described, baseline blood neutrophil counts and mucosal neutrophil counts were obtained on each patient by calculating the mean value of each from a series of repeat measurements taken over a 3-day period prior to the administration of chemotherapy. After the commencement of this therapy, all patients experienced profound neutropenia. Mucosal neutrophil data, expressed as counts and as percentages of baseline counts, were found to coincide with onset of neutropenic fever in 95% of the cases studied and it was found that, in the last group of patients studied, the mucosal neutrophil counts enabled the researchers to predict the onset of neutropenic fever within 24 hours (and in some cases within 12 hours), enhancing the timeliness of initiation of intravenous delivery of broad spectrum antibiotics and thereby averting development of life threatening infection by timely medication.

This study led to the present investigation of developing a rapid test based on a colorimetic assay employing a neutrophil-specific enzymatic reaction that would be simple enough to enable outpatients to self-administer the test, by eliminating the need for time-consuming microscopic counting of neutrophils and substituting a colorimetric intensity readable, e.g., on a simple colorimeter, and correlatable to a predetermined standard relating the neutrophil, enzyme content of the sample to the number of neutrophila present in the sample.

The second group of patients to whom a simple, noninvasive test of this character is potentially attractive is those with HIV-associated neutropenia. Data from a study conducted between 1982 and 1983 on 2047 HIV patients showed significantly higher risks of bacterial infection and need for hospitalization, when blood neutrophil counts declined to less than 750 per cu.mm.(and especially to less than 500 per cu.mm.). When data from 1403 patients with HIV were analyzed, 34.5% developed neutropenia and had increased risk of further developing bacteremia, esophageal candidiasis and bacterial pneumonia. When data from this study were further analyzed in a retrospective matched pair, case-controlled, manner with 24 study individuals vs 8 control persons and 177 patients in each cohort, a relative risk factor of 3.29 and a probability factor of 0.0059 emerged. A higher rate of hospitalization attached to the study individuals, as did an increased mortality rate (38 months median survival vs 135 months for control group). See in this regard (1) Jacobson, M. A., Lui, R. C. and Davies, D. et al “Human immunodeficiency virus disease-related neutropenia and the risk of hospitalization for bacterial infection” Arch. Intern. Med (1997) 157, 1825 and (2) Hermans, P., Sommerejins, B., Van Custem, N., Clumeck, N., “Neutropenia in Patients with HIV Infection: A Case-Controlled Study in a Cohort of 1403 Patients Between 1982 and 1993”, Journal of Hematherapy & Stem Cell Research, 8 (1999) Supp. 1:S23-S32. It is noted, however, that no studies of mucosal neutrophil counts appear to have been made relative to HIV patients with profound neutropenia.

It is clear, nevertheless, based on the available knowledge relating to neutrophil kinetics in the human organism, that access to a noninvasive, simple, quick colorimetric, fluorescent or chemiluminescent test capable of being self administered by outpatients will readily benefit HIV patients who develop profound neutropenia and neutropenic fever.

### BRIEF DESCRIPTION OF THE INVENTION

The present invention employs a modified mouth wash sample collection procedure to obtain the sample which is to be tested. The test involves delivering sample to a test pad on which has been deposited an ester which is known to be cleavable by an enzyme indigenous to neutrophils such as napththol-AS chloroacetate esterase or the characteristic elastase enzyme present in human neutrophils, also known as human leukocyte elastase. This cleavable ester may be so selected that one of its cleavage products possesses a measurable color, fluorescence or chemiluminescence, or it may be codeposited or coimpregnated on a test pad with a compound that spontaneously reacts with a cleavage product to produce color, fluorescence or chemiluminescene. In either case the endpoint of the reaction may be measured electrically by change in current or electrical charge or a color change may be measured by reflectance or absorbance, or changes in fluorescence or chemiluminescence may each be measured by instruments and methods that are also well known in the art.

Any of these measurements can be made with simple instruments that are increasingly readily available for such purposes. Correlation of the measurements so obtained to standards that relate observed measurement intensity values to the concentration of human leukocyte enzyme present in samples, and correlation of enzyme content to number of neutrophils present are both achievable. The latter involves comparin, intensity values obtained in the test herein described when it is practiced on samples of known neutrophil count and establishing a correlation curve. Work needed to effect this standard is in progress. Once completed, such correlations can and will be supplied to outpatients and their support groups in the forms of correlation tables, correlation curves, computer programs, etc., as choice may dictate, to enhance the self-administration and nonprofessional or semi professional administration of the tests and enhance understanding of the test results.

A feature of this invention that lends it greater precision than can be achieved by microscopic counting of neutrophils is that the sample size to be actually measured in each test considerably exceeds that of the two duplicate drops from a suspension of neutrophils and unavoidably associated oral cellular accompaniments (which always include a variable quantity of epithelial cells, microbial flora and cellular debris) upon which neutrophil counts were made by Akpek et al as described at p. 14 of the J. Am. Hematol. paper cited above. Currently ongoing testing to ascertain details of the sample size to be specified in test kits designed to be utilized by persons lacking laboratory training exhibits highly promising trends relative to the outstanding precision and accuracy of this test as herein described and envisioned to be further refined. While the present test is aimed at persons without laboratory training, it is envisioned that its specificity, precision and accuracy may render its use attractive to professionals working in hospital settings and enable elimination of many instances of counting neutrophils under a microscope that are now required in the treatment of iatroginic neutropenia.

### BRIEF DESCRIPTION OF THE DRAWING

Figure is a typical “dose response” curve constructed using mouthwash samples obtained from a single volunteer at different dilutions FIGS. 2 and 3 are plots of % reflectance (“Ro”) against day of testing for 3-day studies using mouthwash samples obtained from 4 healthy volunteers. FIG. 2 employed dilutions containing 2% mouth wash as hereinafter further described and FIG. 3 involved dilutions containing 4% mouthwash.

### DETAILED DESCRIPTION OF THE INVENTION

This invention depends upon the discovery set forth in the Akpek et al paper cited above, that the heretofore widely utilized and widely relied upon method of using repeated blood neutrophil counts of patients treated with anticancer chemotherapy (and especially high dose chemotherapy) as a guide to determining the onset, severity and duration of iatrogenic (or “profound”) neutropenia, including the emergence of neuutropenic fever, is markedly inferior to making repeated measurements of mucosal neutrophil counts. The strong correlation shown to exist between nadirs of neutropenia and mucosel neutrophil counts, and the correlation between the onset and resolution of neutropenic fever with what mucosal neutrophil counts shows, as opposed to blood neutrophil counts, provides a far more reliable and precise basis for confidently pulling patents through bouts of neutropenic fever than has heretofore been available. The benefit of substituting mucosal neutrophil counts for blood neutrophil counts is yet to be tested with those HIV patients who are resistant to protease inhibitors and prone to suffering similar neutropenic attacks, but there is every reason to predict with utmost confidence that the substitution will be at least equally beneficial to them.

Hand-in-hand with the importance of mucosal neutrophil counts to treatment of profound neutropenia, including neutropenic fever, is the need to provide a simple test for mucosal neutrophils that can be availed of outside as well as inside the hospital environment and can be run by patients themselves and/or by their family members, their non-specialist primary care physicians and their office assistants, private duty nurses and nurse assistants caring for patients in the home, and the like.

The most demanding part of the test herein disclosed is the collection of a suitable sample. Mucosal surfaces, in general, are not ready sources of samples and obtaining an adequate sample, (which is of paramount importance to the successful performance of any test important to diagnosis of human physical disease or abnormality), is particularly important here.

In the work reported in their 1986 Blood article cited above, coinventor Wright and coworkers selected a modified mouth wash method for quantifying neutrophils from the oral mucosa based on previous reported experience in using a similar sampling technique for measuring severity of periodontal disease in individuals having normal hematology. Their experience as reported in the Blood article, and the further recent work of coinventor Wright with Akpek et al reported in Am. J. Hematol., supra, show that reliable mucosal neutrophil counts are attainable from mouthwash samples. In the work discussed herein, a modified mouth wash sampling technique was employed. It will be recognized that many other mouth wash formulations could be adopted than that specifically utilized here, that different sample sizes could be adopted and that the description given hereof the mouthwash composition and procedure for using it to collect oral mucosal samples is not intended to be and is not, in any way limiting. It is pointed out further, however, that in any patient regimen or test to be conducted, it will be essential that a standardized composition and procedure for obtaining the mouthwash sample be established and rigorously adhered to throughout so that tests run on the same individual on different days can be confidently compared.

In the work herein discussed, a sterile 0.9% wt/vol saline solution was prepared and buffered with 50 mM of sodium bicarbonate. 10 ml. aliquots of this mixture were measured into sterile 15 ml. conical tubes and distributed to the persons to be tested, who each rinsed their mouths by holding this aliquot in the mouth, with swirling, for a measured 30 seconds and then disposing of the rinse as waste. Each person then waited a timed 15 minutes, while refraining from eating or drinking, and was then given a second 15 ml. conical tube containing another 10 ml. aliquot of the saline/bicarbonate mouthwash buffer referred to in the first sentence. Each then swirled the second aliquot in his or her mouth for a measured 30 seconds and then disgorged it back into the tube. The tubes were labelled with identifying numbers for each person and subjected to testing as described below.

The presence of neutrophils in urine has long been known to be an excellent indicator of the presence of a urinary tract infection and diagnostic assays for detecting urinary infections that apply this knowledge are well known. Test strips developed for the purpose of detecting neutrophils in bodily fluids, with special emphasis on urine, are commercially available. In general, they comprise at least one pad impregnated with a one compound known to be cleaved by a characteristic human neutrophil enzyme, positioned on a strip. Where the cleavage process does not yield a chromogenic product, a dye precursor is impregnated in the same pad as the cleavable compound or in one adjacent to it on the strip, and the dye precursor reacts with at least one cleavage product to form a colored product.

### EXAMPLE 1

A number of the commercially available strips for detecting neutrophils in human bodily fluids were obtained from various sources. Test samples of saline/bicarbonate mouth wash obtained from healthy volunteer human subjects by the method described above were diluted with fresh saline/bicarbonate solution to 2% by volume. Test strips were immersed in these so—diluted samples and color formation was observed on each of the strips. Based on this visual screening, a Hofmann-LaRoche Chemstrip 2LN was selected for further testing based upon its observed uniform and strong color development. Further testing was then conducted using this strip.

According to the manufacturer's information accompanying the Chemstrip 2LN strips, each test pad is impregnated with the following reagent composition per square centimeter of pad surface:

| | | |
|---|---|---|
| Indoxylcarbonic ester | 15.5 | μg |
| Diazonium salt | 5.5 | μg |
| Buffer | 2416.0 | μg |
| Inert ingredients | 2138.0 | μg |

The indoxylcarbonic ester belongs to a class of compounds known to be cleavable by the characteristic human neutrophil elastase enzyme and the diazonium salt is a dye precursor, in this case of a purple color that forms when the dye precursor reacts with a cleavage product from the ester.

A simple reflectance meter equipped with a light source capable of transmitting light at a 580 nm wavelength onto a surface was paired with a detector capable of simultaneously measuring, at 580 nm, the amount of light reflected from the surface. By using this system, the purple-color produced on the neutrophil detection pad absorbs light at the 580 nm wavelength and the increase in colored product is detected as a decrease in reflectance at the same wavelength. After trial runs, a holder was constructed to position the detection pad reproducibly over the light source/detector system.

Thereupon a range of dilutions of mouthwash samples obtained as explained above, from multiple volunteers, were tested over multiple days, to determine sensitivity, precision and accuracy of the test method.

In the course of testing, it was found that timing the reflectance measurement at 5 minutes from the initial appearance of purple color on the strip gave sensitive, precise and accurate values. This can be seen in the Raw Data of Table 1A, in which each “% Ro” figure is the mean of two figures obtained by analyzing replicated dilutions of a mouthwash sample from a normal healthy volunteer on this system. In Table 1A, “MW %” indicates the % of mouthwash in the dilution analyzed and “% Ro@5 min.” is the measured reflectance after 5 minutes from initial appearance of color on the strip. The column headed “stats” denotes calculated values, for each of the mouthwash dilution levels measured as shown in the “MW %” (i.e. Mouthwash %) column of (1) the “mean” value of the measured reflectances in the second column, (2) the “sd” or standard deviation calculated from the measured reflectance values at the same dilution level and (3) the “% CV” or coefficient of variation calculated from the measured reflectance values at the same dilution level.

TABLE 1A
Raw Data Analysis

| MW % | % R₀ @ 5 min | Stats | |
|---|---|---|---|
| 10 | 53.9% | Mean | 55.0% |
| 10 | 55.5% | sd | 1.0% |
| 10 | 55.7% | % CV | 1.7% |
| 6 | 63.9% | Mean | 63.2% |
| 6 | 64.3% | sd | 1.5% |
| 6 | 61.5% | % CV | 2.4% |
| 4 | 72.3% | Mean | 72.6% |
| 4 | 73.6% | sd | 0.9% |
| 4 | 71.8% | % CV | 1.3% |
| 2 | 81.8% | Mean | 82.0% |
| 2 | 82.4% | sd | 0.3% |
| 2 | 81.8% | % CV | 0.4% |
| 1 | 86.3% | Mean | 86.5% |
| 1 | 86.3% | sd | 0.3% |
| 1 | 86.8% | % CV | 0.3% |

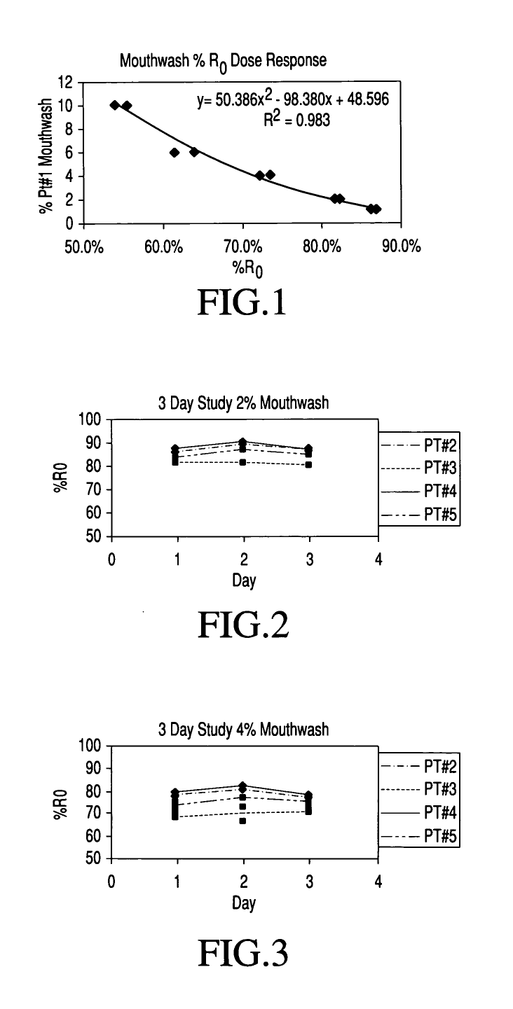

From the values given in Table 1A a standard curve, depicted in FIG. 1 hereof, was plotted, of the percent mouthwash dilution against the percent “Ro”—i.e. the measured % of reflectance—after 5 minutes from the appearance of color on the strip. In the legend of FIG. 1, the symbol “Pt.#1” “Participant 1”, the person whose mouthwash sample was used in making the dilutions for which reflectance values were measured.

The equation of the standard curve shown in FIG. 1, is
y=5.04E+0.1x²−9.84E+0.1x4.86E+01
In this equation, “y” stands for the % mouthwash present in the dilution by volume and the symbol “x” stands for the % reflectance measured at 5 minutes from the first appearance of color on the strip. The symbol “E” as used in the equation represents a factor of 10; while E+01 means 10 to the first power and E−01 is 10⁻¹. R² the correlation coefficient of the curve, is as 9.83 E−01, or 0.983, which represents a high degree of correlation to the data.

The curve of FIG. 1 was utilized to develop the “Interpolated Data Analysis” shown in Table 1B. In Table 1B the FIG. 1 curve was consulted to read the “interpolated” % of mouthwash by volume in the dilution corresponding to each measured % reflectance value that appears in Table 1A. Thus, for example, the “interpolated” % of mouthwash by volume for each of the three measured reflectance values at the actual value of 10% by volume mouthwash present in the dilution are respectively 10.18, 9.51 and 9.43 as shown in the column of Table 1B that is headed “Interpolated % Mouthwash”. The column headed “Error” in Table 1B is the percentage by which the “Interpolated % MW” is above or below the actual value for percent of mouthwash sample present, by volume, in the measured sample. Thus for the first three actual samples tested, each containing 10% by volume of mouthwash, the interpolated volume of 10.18% shows a positive 1.8% error, the interpolated volume of 9.51 exhibits a negative 4.51% error and the interpolated volume of 9.43 represents a −5.7% error. In this Table 1B, the “mean” value shown was calculated from the three “Interpolated % MW” figures for each actual dilution and the “sd” or standard deviation is likewise calculated from the “Interpolated % MW” values given for each actual dilution level, as is the “% CV” or coefficient of variation figure shown.

TABLE 1B
Interpolated Data Analysis

| | % R₀ @ | Interpolated | | | |
|---|---|---|---|---|---|
| % MW | 5 min. | % MW | Error | | |
| 10 | 53.9% | 10.18 | 1.8% | Mean | 9.71 |
| 10 | 55.5% | 9.51 | −4.9% | sd | 0.41 |
| 10 | 55.7% | 9.43 | −5.7% | % CV | 4.3% |
| 6 | 63.9% | 6.30 | 5.0% | Mean | 6.54 |
| 6 | 64.3% | 6.17 | 2.8% | sd | 0.53 |
| 6 | 61.5% | 7.15 | 19.1% | % CV | 8.1% |
| 4 | 72.3% | 3.80 | −4.9% | Mean | 3.74 |
| 4 | 73.6% | 3.48 | −13.0% | sd | 0.23 |
| 4 | 71.8% | 3.93 | −1.7% | % CV | 6.2% |
| 2 | 81.8% | 1.83 | −8.4% | Mean | 1.80 |
| 2 | 82.4% | 1.74 | −13.1% | sd | 0.05 |
| 2 | 81.8% | 1.83 | −8.4% | % CV | 3.0% |
| 1 | 86.3% | 1.22 | 21.7% | Mean | 1.20 |
| 1 | 86.3% | 1.22 | 21.7% | sd | 0.03 |
| 1 | 86.8% | 1.16 | 16.1% | % CV | 2.7% |

The purpose of reading the “Interpolated Data” of Table 1B from the standard curve and calculating “Error”, “mean”, “sd” and “% CV” for each actual sample dilution is, as those skilled in the art will understand, to subject the measured raw data and the excellent correlation coefficient of the curve drawn based on the raw data to further challenge.

### EXAMPLE 2

Using the strip selected in Example 1, the sample collection method described earlier and the measurement methodology established in Example 1, series of test runs were made to establish the day to day stability of individual baseline oral mucosal neutrophil concentration in a normal healthy individual. In these runs, the mouthwash samples, taken as described herein, were obtained on six separate days over a time period spanning two weeks at exactly the same time of day. They were then measured in dilutions each containing 2% by volume of mouthwash sample.

Table 2 below shows the measured reflectance results for each test day. The calculated “mean”, standard deviation and coefficient of variation figures for the test series reflects extreme stability of baseline neutrophil content in the oral mucosa of this individual.

TABLE 2
% of Initial Reflectance: PT #1 @ 2%

| | | |
|---|---|---|
| Date | % R₀ | Overall Precision |

| | | | |
|---|---|---|---|
| Feb. 2, 2004 | 77.6% | Mean | 81.0% |
| | 82.5% | sd | 1.8% |
| | 76.3% | CV | 2.2% |
| Feb. 4, 2004 | 81.7% | | |
| | 82.3% | | |
| | 80.7% | | |
| Feb. 10, 2004 | 82.0% | | |
| | 80.8% | | |
| | 79.8% | | |
| Feb. 11, 2004 | 82.9% | | |
| | 81.0% | | |
| | 80.7% | | |
| | 79.4% | | |
| Feb. 12, 2004 | 81.8% | | |
| | 82.4% | | |
| | 81.8% | | |
| Feb. 13, 2004 | 82.5% | | |

### EXAMPLE 3

Two similar studies of the oral mucosal neutrophil levels, each over three consecutive days, were made on mouthwash samples, collected as described above, from 4 different volunteers designated PT #2, PT #3, PT #4 and PT #5, (where “PT” means Participant).

In the first study, the samples on which the measurements were made were dilutions each containing 2% by volume of mouthwash. The tests were performed on the chemstrip 2LN strips employed in Example 1 and were conducted in the same manner as those, results of which appear in Tables 1A and 2 hereof. The measured results in “% Ro” are set forth in Table 3 along with mean reflectance values calculated from duplicate measurements obtained daily on each volunteer over the three day period and calculated values of standard deviation and coefficient of variation for each volunteer's samples.

TABLE 3
Three Day Study of Volunteer Mouthwash 2% Dilution.

| | PT#2 | PT#3 | PT#4 | PT#5 |
|---|---|---|---|---|
| Day | % R₀ | % R₀ | % R₀ | % R₀ |
| 1 | 88.9% | 82.9% | 88.0% | 85.2% |
| | 86.0% | 80.2% | 87.5% | 84.0% |
| 2 | 90.1% | 82.1% | 91.1% | 87.3% |
| | 89.7% | 82.3% | 90.8% | 87.3% |
| 3 | 87.2% | 80.6% | 87.9% | 83.9% |
| | 87.6% | 80.0% | 85.9% | 85.2% |
| Mean | 88.3% | 81.4% | 88.5% | 85.5% |
| sd | 1.6% | 1.2% | 2.0% | 1.5% |
| CV | 1.8% | 1.5% | 2.3% | 1.8% |

Another 3-day study performed at a different time, on mouthwash samples collected daily from each of the same four volunteers was made on dilutions each containing 4% by volume of mouthwash. In this study, too, the collection of mouthwash samples was as described above and the tests were performed in the same manner as those, the results of which appear in Tables 1A, 2 and 3 hereof. Data from this study appear in Table 4 along with a mean value of measured % Ro for calculated for each test participant and calculated values of standard deviation and coefficient of variation for each participant's test results obtained in this series.

FIG. 2 hereof is a graphic representation of the measured % Ro over 3 consecutive days for sample dilutions each containing 2% mouthwash from individual participant samples obtained daily over the three day period, while FIG. 4 is a similar graph of measured % Ro over three consecutive days for sample dilutions, each containing 4% mouthwash, from samples that each participant gave daily over the 3-day period of the study. In the two graphs, FIGS. 3 and 4, the measured Ro values obtained on samples from each of the four participants are each depicted in a different color from the color depicting the measured Ro values for each other participant. The individual lines in both studies show a high degree of stability in baseline mucosal neutrophil concentration for each individual, as well as some degree of variation in the baseline itself among the four individuals. Both of these results are expected and both attest to the precision and accuracy of the test.

The foregoing description itself shows, and those skilled in the art of immunochemistry and immunology will readily understand, that numerous changes in measurement methodology, test strip formulation, substance selected to be measured, sample selection, mouthwash formulation and concentration and other parameters discussed herein can be made without departing from the spirit and scope of this invention. It is intended, therefore, that the invention be circumscribed, if at all, only by the scope of the appended claims.

TABLE 4
Three Day Study of Volunteer Mouthwash 4% Dilution.

| | PT#2 | PT#3 | PT#4 | PT#5 |
|---|---|---|---|---|
| Day | % R₀ | % R₀ | % R₀ | % R₀ |
| 1 | 77.6% | 69.4% | 79.5% | 74.3% |
| | 81.6% | 66.4% | 82.4% | 77.8% |
| 2 | 80.6% | 72.5% | 82.5% | 76.6% |
| | 76.5% | 72.2% | 78.9% | 75.4% |
| 3 | 76.8% | 69.4% | 76.1% | 75.2% |
| | 78.7% | 69.5% | 79.6% | 75.3% |
| Mean | 78.6% | 69.9% | 79.8% | 75.8% |
| sd | 2.1% | 2.2% | 2.4% | 1.2% |
| CV | 2.6% | 3.2% | 3.0% | 1.6% |

1. A method for determining the mucosal neutrophil count with an exceptional degree of sensitivity, accuracy and precision of a human individual which comprises the steps of
(a) obtaining from said person an oral mucosal sample collected by swishing a measured quantity of a mouthwash around his or her mouth for a timed interval of not more than 1 minute,
(b) delivering a measured quantity of said sample or of an aqueous dilution of said sample to a sample pad supported on a strip, said sample pad having impregnated therein or deposited thereon
(i) a sufficient quantity of a chemical compound that is cleaved into at least two fragments when exposed to the action of an enzyme known to be characteristically present in human neutrophils to react completely with the target enzyme in the sample and
(ii) unless one of said fragments is inherently chromophoric, a dye precursor, a fluorescence imparting material or a chemilumines imparting compound in sufficient quantity to react completely with one of said fragments to form a colored, fluorescent or chemiluminescent product,
(c) allowing said sample or said aqueous dilution of sample and the ingredients present on the pad to remain in contact as color, fluorescence or chemiluminescence develops
(d) measuring the intensity of the color, fluorescence or chemiluminescence and
(e) comparing the intensity measured to a predetermined standard that correlates the measurement to the number of human neutrophils in the sample.

2. A method according to claim 1 wherein the mouth wash in step (a) is a saline/bicarbonate buffer, the compound of step b (i) is an ester cleavable by human leukocyte elastase and the ingredient of step b (ii) is a dye that reacts with one of said fragments to form a distinct color.

3. A method according to claim 2 wherein the compound of step (b) is an indoxylcarbonic acid ester and the dye of step b (ii) is a diazo dye that produces a uniform purple color.

4. A method accordingly to claim 3 wherein the mouthwash sample is collected after a times interval of 30 seconds.
